(19) United States
(12) Patent Application Publication
(10) Pub. No.: US 2007/0105204 A1
(43) Pub. Date: May 10, 2007

(54) Methods for producing propylene glycol monoesters using a lipase

(75) Inventors: Wuli Bao, Champaign, IL (US); Leif Solheim, Decatur, IL (US)

Correspondence Address: STERNE, KESSLER, GOLDSTEIN & FOX P.L.L.C., 1100 NEW YORK AVENUE, N.W., WASHINGTON, DC, 20005, US

(73) Assignee: Archer-Daniels-Midland Company, Decatur, IL (US)

(21) Appl. No.: 11/598,106

(22) Filed: Nov. 13, 2006

Related U.S. Application Data
(60) Provisional application No. 60/735,150, filed on Nov. 10, 2005.

(51) Int. Cl. A61K 36/00; C12P 7/02

(52) U.S. Cl. 435/155; 424/725

(57) ABSTRACT

The present invention relates to methods for producing propylene glycol monoesters (PGMEs). A method comprises contacting a fatty acid material with a lipase to form a first composition, contacting the first composition with propylene glycol to form a second composition, and heating the second composition, wherein a PGME composition is produced. The present methods yield between about 40% to about 70% PGMEs or greater, and less than about 15% propylene glycol diester.

### CROSS REFERENCE TO RELATED APPLICATIONS

This application claims the benefit of U.S. Provisional Application No. 60/735,150 filed Nov. 10, 2005, which is incorporated by reference herein in its entirety.

### BACKGROUND OF THE INVENTION

1. Field of the Invention

The invention relates to methods for producing propylene glycol monoesters (PGMEs) using a lipase.

Propylene glycol monoesters containing a natural or synthetic fatty acid moiety can be obtained via chemical or enzymatic transesterification or esterification processes. By these processes, the fatty acyl portion of a fatty acid, generally derived from an oil or fat, is transferred to one of the hydroxyl moieties of the propylene glycol molecule.

In general, the preparation of PGMEs is possible from a number of routes. For example, propylene glycol and triglycerides can be reacted together using an alkaline catalyst to give a reaction product comprising monoesters of propylene glycol, propylene glycol diesters, monoglycerides, diglycerides, and triglycerides, after removal of the excess propylene glycol and glycerol (Hui, Y. H., “Manufacturing Processes for Emulsifiers” in Bailey's Industrial Oils and Fat Products, John Wiley & Sons, Inc. 5^{th} Ed., Vol. 4, pp. 569-601 (1996)). The most commonly utilized process of making propylene glycol monoesters is by interesterifying triglycerides with propylene glycol. This interesterification reaction proceeds at temperatures ranging from 350° to 450° F. with the use of a catalyst such as sodium hydroxide. The resulting crude product contains propylene glycol mono- and diesters, monoglycerides and diglycerides, as well as numerous by-products. The final product composition of these processes can be described in terms of the ratio of mono- to diesters comprising the product. The composition of the end product can be controlled by varying the amounts of polyol with respect to oil, and through manipulating the reaction conditions. A higher concentration of monoesters is usually obtained through a molecular distillation process.

A second route is through the reaction of propylene glycol with fatty acids or fatty acid esters, such as methyl or ethyl esters of fatty acids (Swern, D., “Fat Splitting, Esterification, and Interesterification” in Bailey's Industrial Oils and Fat Products, John Wiley & Sons, Inc., 4^{th} Ed., Vol. 2, pp. 97-173 (1982)). Direct esterification under practical conditions can be accomplished by reacting propylene glycol with a fatty acid to yield approximately 55 to 60 percent of a propylene glycol monoester product; the balance is a reaction by-product comprising diester and unreacted starting material. Because of the high cost of fatty acids relative to triglycerides, the direct esterification process is not commonly utilized. An acid such as para-toluene sulfonic acid catalyzes the esterification of palmitic acid and propylene glycol (see, e.g., U.S. Pat. No. 3,669,848). Reaction of fatty acid methyl esters with glycols was accomplished with the addition of metallic sodium as catalyst and the evolution of methanol. The product from these reactions will generally be a mixture comprising primarily mono- and diesters of propylene glycol after the removal of water or the low-boiling alcohol (ethanol, methanol, etc.) by-products and any excess starting reactants.

A third route is combining propylene oxide with fatty acid, leading to a mixture of monoester isomers.

A fourth route is combining propylene glycol with an acid chloride of a fatty acid.

The above chemical esterification or transesterification methods require costly chemicals, high temperatures and generate wasteful by-products. The composition (relative percent of monoesters to diesters) of the end product can be controlled in a limited way by varying the amounts of glycol with respect to fatty acid reagent (methyl ester, fatty acid or oil), and through manipulating the reaction conditions. The above reaction processes, however, consistently generate color during the preparation of the propylene glycol fatty acid ester product. It is desirable to prepare a propylene glycol monoester mixture of acceptable color. A dark-colored monoester mixture is not suitable for incorporation into products such as paint or food. Further, it is desirable to prepare a propylene glycol monoester mixture that has a low percentage of diesters.

Another method of producing a PGME involves the use of an enzyme. In contrast to chemical methods, enzymatic methods of transesterification or esterification are simpler, cleaner and more environmentally friendly. The final product composition of enzymatic processes (also called “propylene glycol monoester composition”) can also be described in terms of the ratio of mono- to diesters comprising the product.

The enzyme capable of affecting the transesterification or esterification is known as a lipase (Triacylglycerol acylhydrolases, EC 3.1.1.3). Lipases are obtained from prokaryotic or eukaryotic microorganisms and typically fall into one of three categories (Macrae, A. R., J.A.O.C.S. 60: 243A-246A (1983)).

The first category includes nonspecific lipases capable of releasing or binding any fatty acid from or to any glyceride position. These lipases provide little selectivity over chemical processes. Such lipases have been obtained from Candida cylindracae, Corynebacterium acnes and Staphylococcus aureus (see, e.g., Macrae, A. R., J.A.O.C.S. 60:243A-246A (1983) and U.S. Pat. No. 5,128,251). The second category of lipases only adds or removes specific fatty acids to or from specific glycerides. Thus, these lipases are only useful in producing or modifying specific glycerides. Such lipases have been obtained from Geotrichum candidium and Rhizopus, Aspergilus, and Mucor genera (see, e.g., Macrae, A. R., J.A.O.C.S. 60:243A-246A (1983) and U.S. Pat. No. 5,128,251). The last category of lipases catalyze the removal or addition of fatty acids from the glyceride carbons on the end in the 1- and 3-positions. Such lipases have been obtained from Thermomyces lanuginosa, Rhizomucor miehei, Aspergillus niger, Mucor javanicus, Rhizopus delemar, and Rhizopus arrhizus (see, e.g., Macrae, A. R., J.A.O.C.S. 60:243A-246A (1983)).

Despite the benefits of using a lipase, methods of enzymatic transesterification or esterification to yield PGMEs may produce lower amounts of PGMEs in the final product than chemical methods. It would therefore be desirable to improve the enzymatic processes to increase the efficiency of the process and increase the half-life of the enzyme.

### SUMMARY OF THE INVENTION

The invention relates to a method for producing a propylene glycol monoester (PGME) composition comprising (a) contacting a fatty acid material with a lipase to form a first composition; (b) contacting the first composition with propylene glycol to form a second composition; and (c) heating the second composition at a temperature from about 10° C. to about 90° C.; wherein a propylene glycol monoester composition is produced.

Optionally, the fatty acid material in step (a) and/or the first composition is heated at a temperature from about 10° C. to about 90° C., and heating is continued through step (c).

The invention also relates to a method for producing a propylene glycol monoester composition comprising (a) heating a fatty acid material at a temperature from about 10° C. to about 90° C.; (b) contacting the heated fatty acid material with a lipase to form a mixture; and (c) contacting the mixture with propylene glycol; wherein a propylene glycol monoester composition is produced.

The present methods yield from about 40% to about 70% PGMEs, or greater. Another feature of the present methods is that the reactants and mixtures thereof do not require solvation in an organic solvent.

### DETAILED DESCRIPTION OF THE INVENTION

In one embodiment, the invention relates to a method for producing a propylene glycol monoester (PGME) composition. The method comprises (a) contacting a fatty acid material with a lipase to form a first composition, (b) contacting the first composition with propylene glycol to form a second composition, and (c) heating the second composition, wherein a propylene glycol monoester composition is produced.

The fatty acid material in step (a) is optionally heated prior to contacting with a lipase, and heating is continued through steps (a) to (c). Alternatively, the first composition comprising the fatty acid material/lipase mixture is heated prior to step (b), and heating is continued through steps (b) and (c). Thus, step (c) encompasses continued heating of the second composition when the fatty acid material and/or the first composition has already been heated.

In another embodiment, the invention relates to a method for producing a propylene glycol monoester composition comprising: (a) heating a fatty acid material; (b) contacting the heated fatty acid material with a lipase to form a mixture; and (c) contacting the mixture with propylene glycol; wherein a propylene glycol monoester composition is produced. Optionally, steps (b) and (c) of this method can be conducted while heating.

In a preferred embodiment, the method comprises heating the fatty acid material, contacting the heated fatty acid material with the lipase while heating to form a fatty acid material/enzyme mixture, and contacting the mixture with propylene glycol while heating to form a propylene glycol monoester composition.

The heating temperature of the fatty acid material, the fatty acid material/lipase mixture (the first composition), and/or the second composition in the process can be the same or different. Useful heating temperatures in the process, including heating of the fatty acid material, the fatty acid material/lipase mixture (the first composition), and/or the second composition can be from about 10° C. to about 90° C. Preferably, the temperature is from about 30° C. to about 70° C. Most preferably, the temperature is from about 40° C. to about 60° C., or about 50° C.

The present invention also encompasses modifying the above methods to include one or more sparges with an inert gas, operation of the above methods in contact with an inert gas atmosphere, and/or contacting the fatty acid material/lipase mixture with propylene glycol under a vacuum. Such modifications can increase the yield of PGMEs. Thus, an embodiment, the present methods further comprise sparging the initial fatty acid material/lipase mixture with an inert gas. The inert gas can be any inert gas such as Ar or N₂. In another embodiment, the fatty acid/lipase mixture can be contacted with propylene glycol under a vacuum or inert atmosphere. The vacuum can be from about 1 mm Hg to about 15 mm Hg.

The yield of the PGME product resulting from the methods of the invention is surprisingly high, particularly when the propylene glycol is added after the fatty acid material and lipase have been contacted. Thus, the propylene glycol is preferably added to the mixture of the fatty acid material and lipase. The propylene glycol can be added at a rate per minute of about 1% to about 10% of the total weight of the propylene glycol to be added.

In another embodiment of the invention, the reaction can proceed essentially in the absence of additional organic solvent. In this embodiment, none of the reactants have been appreciably solvated by organic solvents prior to use in the present method. The reactants are used essentially in their neat state. Furthermore, the methods of the invention can be conducted without the addition of organic solvent to the mixtures and products described above (e.g., the fatty acid material, lipase, propylene glycol, or mixtures or products thereof).

The term “organic solvent” is meant to include those solvents referred to in the field as organic, and excludes water, reactants such as propylene glycol, and any by-products produced during the reaction such as methanol.

Since the present methods do not require the use of organic solvents, in an embodiment the method specifically excludes the use of any organic solvent to solvate a reactant prior to combining, or the combining of an organic solvent to the mixtures and products described in the methods above.

As the reactants need not be solvated in an additional organic solvent, the present methods are especially suited for producing PGMEs for use in foods. Also, PGMEs can be produced more economically because the costs of adding an organic solvent during reaction followed by removing the solvent from the product are avoided. Furthermore, in the final product there is no residual organic solvent which would have to be exhaustively removed or would otherwise limit the usefulness of the final PGME.

As stated above, the present methods provide an excellent percent product yield. The present methods, whether incorporating sparging, vacuum step(s) and/or an inert atmosphere, produce a PGME composition comprising at least about 40% PGMEs by weight of the composition. Preferably, the method yields a composition comprising at least about 60% PGMEs. More preferably, the methods yield a composition comprising at least about 70% PGMEs, or greater; for example, about 75%. Illustratively, the present methods yield between about 40% to about 70% PGMEs, or about 50% to about 70% PGMEs, or about 60% to about 70% PGMEs, or about 65% to about 70% PGMEs, or about 70% PGMEs.

Yet another feature is that the present methods do not add undesirable color to the PGME product. Darkening of the product in comparison to the color of the starting material is an undesirable feature of chemical methods. The present methods, on the other hand, produce PGMEs that are essentially the same color as the fatty acid starting material. Thus, the present methods are especially desirable for producing PGMEs whose end product use is in foods or paints. Color is typically measured on the Lovibond color scale using a 5.25 inch cell. Useful color values of PGMEs for food or industrial uses are less than or equal to about 3.5 yellow and about 1.0 red. Preferably, the Lovibond color is less than about 2.5 yellow and 0.5 red. Most preferably, the Lovibond color is less than about 1.0 yellow and 0.2 red.

The present methods can yield propylene glycol diesters (PGDEs) as a by-product. The amount of PGDEs formed during the reaction is relatively small. The amount of PGDEs present in the PGME product produced by the present method is typically lower than the amount of PGME present. Preferably, the PGDEs are present in an amount lower than about 15% of the total weight of the product. More preferably, the PGDEs are present in an amount less than about 10% of the total weight. Most preferably, the PGDEs are present in an amount less than about 5% of the total weight. Thus, the present methods yields PGME compositions comprising a high percentage of monoesters and only a small percentage of PGDEs as by-products.

Esterification or transesterification as utilized in the present process are the processes by which an acyl group is added, hydrolyzed, repositioned or replaced on a polyhydroxyl alcohol such as propylene glycol. The acyl group can be derived from an ester or free fatty acid. The alkyl moiety is a polyhydroxy alcohol moiety as described above. Transesterification or esterification can be effected by a lipase derived from the microorganism known as Mucor miehei, from the Mucor genus. This lipase is available commercially. One example is Novozyme Lipozyme RM-IM.

Nonspecific lipases capable of releasing or binding any fatty acid from or to any glyceride position. These lipases provide little selectivity over chemical processes. Such lipases have been obtained from Candida cylindracae, Corynebacterium acnes and Staphylococcus aureus (see, e.g., Macrae, A. R., J.A.O.C.S. 60:243A-246A (1983) and U.S. Pat. No. 5,128,251). Another type of lipase only adds or removes specific fatty acids to or from specific glycerides. Thus, these lipases are only useful in producing or modifying specific glycerides. Such lipases have been obtained from Geotrichum candidium and Rhizopus, Aspergilus, and Mucor genera (see, e.g., Macrae, A. R., J.A.O.C.S. 60:243A-246A (1983) and U.S. Pat. No. 5,128,251). A more preferred type of lipase catalyzes the removal or addition of fatty acids from the glyceride carbons on the end in the 1- and 3-positions. Such 1,3-specific lipases have been obtained from Thermomyces lanuginosa, Rhizomucor miehei, Aspergillus niger, Mucor javanicus, Rhizopus delemar, Candida antarctica, and Rhizopus arrhizus (see, e.g., Macrae, A. R., J.A.O.C.S. 60:243A-246A (1983)). Lipase obtained from Candida antarctica is commercially available from Novozymes (Denmark) as Novozym 435.

The lipase obtained from the organisms above can be immobilized for the present invention using suitable carriers by a usual method known to persons of ordinary skill in the art. U.S. Pat. Nos. 4,798,793; 5,166,064; 5,219,733; 5,292,649; and 5,773,266 describe examples of immobilized lipase and methods of preparation. Examples of methods of preparation include the entrapping method, inorganic carrier covalent bond method, organic carrier covalent bond method, and the adsorption method. The lipases used in the Examples below were obtained from Novozymes (Denmark) but can be substituted with purified and/or immobilized lipase prepared by others. The present invention also contemplates using crude enzyme preparations or cells of microorganisms capable of expressing or overexpressing lipase, a culture of such cells, a substrate enzyme solution obtained by treating the culture, or a composition containing the enzyme.

U.S. Pat. Nos. 4,940,845 and 5,219,733 describe the characteristics of several useful carriers. Useful carriers are preferably microporous and have a hydrophobic porous surface. Usually, the pores have an average radius of about 10 Å to about 1,000 Å, and a porosity from about 20 to about 80% by volume, more preferably, from about 40 to about 60% by volume. The pores give the carrier an increased enzyme bonding area per particle of the carrier. Examples of preferred inorganic carriers include porous glass, porous ceramics, celite, porous metallic particles such as titanium oxide, stainless steel or alumina, porous silica gel, molecular sieve, active carbon, clay, kaolinite, perlite, glass fibers, diatomaceous earth, bentonite, hydroxyapatite, calcium phosphate gel, and alkylamine derivatives of inorganic carriers. Examples of preferred organic carriers include microporous Teflon, aliphatic olefinic polymer (e.g., polyethylene, polypropylene, a homo- or copolymer of styrene or a blend thereof or a pretreated inorganic support), nylon, polyamides, polycarbonates, nitrocellulose and acetylcellulose. Other suitable organic carriers include hydrophillic polysaccharides such as agarose gel with an alkyl, phenyl, trityl or other similar hydrophobic group to provide a hydrophobic porous surface (e.g., “Octyl-Sepharose CL-4B”, “Phenyl-Sepharose CL-4B”, both products of Pharmacia Fine Chemicals). Microporous adsorbing resins include those made of styrene or alkylamine polymer, chelate resin, ion exchange resin such a “DOWEX MWA-1” (weakly basic anion exchange resin manufactured by the Dow Chemical Co., having a tertiary amine as the exchange group, composed basically of polystyrene chains cross linked with divinylbenzene, 150 Å in average pore radius and 20-50 mesh in particle size), and hydrophilic cellulose resin such as one prepared by masking the hydrophilic group of a cellulosic carrier, e.g., “Cellulofine GC700-m” (product of Chisso Corporation, 45-105 μm in particle size).

The fatty acid starting materials include any fatty acid material that is an appropriate substrate for the enzyme. A fatty acid material includes fatty acids and derivatives thereof such as esters of fatty acids. Examples of fatty acids useful in the present invention include saturated straight-chain or branched fatty acids, unsaturated straight-chain or branched fatty acids, hydroxy fatty acids, and polycarboxylic acids. The fatty acids can be naturally occurring, processed or refined from natural products or synthetically produced. Although there is no upper or lower limit for the length of the longest carbon chain in useful fatty acids, it is preferable that their length is about 6 to about 34 carbons long. Specific fatty acids useful for the present invention are described in, e.g., U.S. Pat. Nos. 4,883,684; 5,124,166; 5,149,642; 5,219,733; and 5,399,728.

Examples of useful saturated straight-chain fatty acids having an even number of carbon atoms include acetic acid, butyric acid, caproic acid, caprylic acid, capric acid, lauric acid, myristic acid, palmitic acid, stearic acid, arachic acid, behenic acid, lignoceric acid, hexacosanoic acid, octacosanoic acid, triacontanoic acid and n-dotriacontanoic acid, and those having an odd number of carbon atoms, such as propionic acid, n-valeric acid, enanthic acid, pelargonic acid, hendecanoic acid, tridecanoic acid, pentadecanoic acid, heptadecanoic acid, nonadecanoic acid, heneicosanoic acid, tricosanoic acid, pentacosanoic acid and heptacosanoic acid (see, e.g., U.S. Pat. No. 5,219,733).

Examples of useful saturated branched fatty acids include isobutyric acid, isocaproic acid, isocaprylic acid, isocapric acid, isolauric acid, 11-methyldodecanoic acid, isomyristic acid, 13-methyl-tetradecanoic acid, isopalmitic acid, 15-methyl-hexadecanoic acid, isostearic acid, 17-methyloctadecanoic acid, isoarachic acid, 19-methyl-eicosanoic acid, α-ethyl-hexanoic acid, α-hexyldecanoic acid, a-heptylundecanoic acid, 2-decyltetradecanoic acid, 2-undecyltetradecanoic acid, 2-decylpentadecanoic acid, 2-undecylpentadecanoic acid, and Fine oxocol 1800 acid (product of Nissan Chemical Industries, Ltd.) (see, e.g., U.S. Pat. No. 5,219,733).

Examples of useful saturated odd-carbon branched fatty acids include anteiso fatty acids terminating with an isobutyl group, such as 6-methyl-octanoic acid, 8-methyl-decanoic acid, 10-methyl-dodecanoic acid, 12-methyl-tetradecanoic acid, 14-methyl-hexadecanoic acid, 16-methyl-octadecanoic acid, 18-methyl-eicosanoic acid, 20-methyl-docosanoic acid, 22-methyl-tetracosanoic acid, 24-methyl-hexacosanoic acid and 26-methyloctacosanoic acid (see, e.g., U.S. Pat. No. 5,219,733).

Examples of useful unsaturated fatty acids include 4-decenoic acid, caproleic acid, 4-dodecenoic acid, 5-dodecenoic acid, lauroleic acid, 4-tetradecenoic acid, 5-tetradecenoic acid, 9-tetradecenoic acid, palmitoleic acid, 6-octadecenoic acid, oleic acid, 9-octadecenoic acid, 11-octadecenoic acid, 9-eicosenoic acid, cis-11-eicosenoic acid, cetoleic acid, 13-docosenoic acid, 15-tetracosenoic acid, 17-hexacosenoic acid, 6,9,12,15-hexadecatetraenoic acid, linoleic acid, linolenic acid, α-eleostearic acid, β-eleostearic acid, punicic acid, 6,9,12,15-octadecatetraenoic acid, parinaric acid, 5,8,11,14-eicosatetraenoic acid, 5,8,11,14,17-eicosapentaenoic acid (EPA), 7,10,13,16,19-docosapentaenoic acid, 4,7,10,13,16,19-docosahexaenoic acid (DHA) and the like (see, e.g., U.S. Pat. No. 5,219,733).

Examples of useful hydroxy fatty acids include α-hydroxylauric acid, α-hydroxymyristic acid, α-hydroxypalmitic acid, α-hydroxystearic acid, ω-hydroxylauric acid, α-hydroxyarachic acid, 9-hydroxy-12-octadecenoic acid, ricinoleic acid, α-hydroxybehenic acid, 9-hydroxy-trans-10,12-octadecadienic acid, kamolenic acid, ipurolic acid, 9,10-dihydroxystearic acid, 12-hydroxystearic acid and the like (see, e.g., U.S. Pat. No. 5,219,733).

Examples of useful polycarboxylic acids include oxalic acid, citric acid, malonic acid, succinic acid, glutaric acid, adipic acid, pimelic acid, suberic acid, azelaic acid, sebacic acid, D,L-malic acid and the like (see, e.g., U.S. Pat. No. 5,219,733).

Preferably, the free fatty acids have carbon chains from 4 to 34 carbons long. More preferably, the free fatty acids have carbon chains from 4 to 26 carbons long. Most preferably, the free fatty acids have carbon chains from 12 to 26 carbons long. Preferably, the free fatty acids are selected from the following group: palmitic acid, stearic acid, oleic acid, linoleic acid, linolenic acid, arachidonic acid, erucic acid, caproic acid, caprylic acid, capric acid, eicosapentanoic acid (EPA), docosahexaenoic acid (DHA), lauric acid, myristic acid, 5-eicosenoic acid, butyric acid, gamma-linolenic acid and conjugated linoleic acid.

Fatty acids derived from various plant and animal fats and oils (such as fish oil fatty acids) and processed or refined fatty acids from plant and animal fats and oils (such as fractionated fish oil fatty acids in which EPA and DHA are concentrated) can also be added. Medium chain fatty acids (as described by Merolli, A. et al., INFORM 8:597-603 (1997)) can also be used.

In the most preferred embodiment, the fatty acid starting material can be a vegetable oil fatty acid material. Vegetable oil fatty acids are derived from vegetable oils. Preferred vegetable oils include, but are not limited to, soybean oil, linseed oil, sunflower oil, castor oil, corn oil, canola oil, rapeseed oil, palm kernel oil, cottonseed oil, peanut oil, coconut oil, palm oil, tung oil, safflower oil and derivatives, conjugated derivatives, genetically-modified derivatives and mixtures thereof. As used herein, a reference to a vegetable oil includes all its derivatives as outlined above. For instance, the use of the term “linseed oil” includes all derivatives including conjugated linseed oil.

Fatty acids derived from vegetable oils include fatty acids containing carbon chains of about 12 to about 26 carbons. The fatty acid chain can be saturated, unsaturated or polyunsaturated. Preferably, the fatty acid is unsaturated or polyunsaturated. Preferred unsaturated or polyunsaturated fatty acids include, but are not limited to, palmitoleic acid, oleic acid, linoleic acid, linolenic acid, eleostearic acid, ricinoleic acid, arachidonic acid, cetoleic acid or erucic acid.

In a preferred embodiment, the fatty acids are derived from soy oil. A soy fatty acid composition can contain C16:0 Palmitic, C17:0 Margaric, C18:0 Stearic, C18:1 Oleic, C18:2 Linoleic, C18:3 Linolenic, C20:0 Arachidonic, C20:1 Gadoleic, C22:0 Behenic, C24:0 Lignoceric, as well as other fatty acids.

For the present methods, the fatty acids and derivatives thereof can be used singly, or at least two of such acids of the same group or different groups are usable in admixture. A fatty acid material containing at least two chemically different C₁₂₋₂₆ fatty acids or derivatives thereof in an admixture is generally less expensive than a purified fatty acid material, and is suitable for use in the present method. However, a purified or homogenous fatty acid material containing substantially one distinct type of C₁₂₋₂₆ fatty acid or derivative thereof is also suitable.

In a preferred embodiment, the fatty acid derivative is a C₁₋₄ alkyl ester of a fatty acid. More preferably, the fatty acid material is a methyl ester of a vegetable oil fatty acid. When the fatty acid material is an ester, the ester is transesterified by the lipase to yield a PGME composition.

The present invention can be used in batch slurry type reactions in which the slurry of lipases and substrates are mixed vigorously to ensure a good contact between them. Preferably, the transesterification or esterification reaction is carried out in a fixed bed reactor with immobilized lipases.

Lipase enzymatic activity is also affected by factors such as temperature, light and moisture content. Temperature is controlled as described above. Light can be kept out by using various light blocking or filtering means known in the art. Moisture content, which includes ambient atmospheric moisture, is controlled by operating the process as a closed system. The closed system can be under a positive nitrogenous pressure to expel moisture. Alternatively, a bed of nitrogen gas can be placed on top of the substrate, purification bed or column, or packed lipase column. Other inert gasses such as helium or argon can also be used. These techniques have the added benefit of keeping atmospheric oxidative species (including oxygen) away from the substrate, product or enzyme.

Immobilized lipase can be mixed with initial or purified substrate to form a slurry which is packed into a suitable column. Initial substrate is prepared from one or more glycerides, monoglycerides, diglycerides, triglycerides, free fatty acids, monohydroxyl alcohols, polyhydroxyl alcohols and esters. The temperature of the substrate is regulated so that it can continuously flow though the column for contact with the lipase and transesterification or esterification. If solid glycerides or fatty acids are used, the substrate is heated to a fluid state. The substrate can be caused to flow through the column(s) under the force of gravity, by using a peristaltic or piston pump, under the influence of a suction or vacuum pump, or using a centrifugal pump. The transesterified fats and oils produced are collected and the desired glycerides are separated from the mixture of reaction products by methods well known in the art. This continuous method involves a reduced likelihood of permitting exposure of the substrates to air during reaction and therefore has the advantage that unsaturated fatty acids, glycerides or the like, if used, will not be exposed to moisture or oxidative species. Alternatively, reaction tanks for batch slurry type production as described above can also be used. Preferably, these reaction tanks are also sealed from air so as to prevent exposure to oxygen, moisture, or other ambient oxidizing species.

### EXAMPLES

The following examples are illustrative only and are not intended to limit the scope of the invention as defined by the appended claims.

### Example 1

A mixture of fatty acids (50 g) derived from soy was heated at about 50° C. Lipase (Lipozyme RM-IM), 5 g, was added to the fatty acid mixture. The fatty acids and enzyme were then subjected to a vacuum (from about 1 to about 10 mm Hg). To the reaction was added propylene glycol (16 g) over the course of about 30 minutes. A sample of the reaction was taken at the times indicted in Table 1 below.

TABLE 1
Production of PGME from Fatty Acids using Lipozyme RM-IM

Sample Id

| | 78-1 | 78-2 | 78-3 | 78-4 |
|---|---|---|---|---|
| Time (min) | 30 | 60 | 90 | 120 |
| PGME | 57.87 | 70.16 | 74.04 | 75.73 |
| PGDE | 4.56 | 5.6 | 6.1 | 7.53 |
| FA | 30.53 | 17.92 | 13.1 | 9.93 |
| PG | 3.26 | 5.84 | 7.57 | 6.83 |
| Total | 96.22 | 99.52 | 100.81 | 100.02 |

### Example 2

A mixture of fatty acids (50 g) derived from soy having color values of about 2.4 yellow and about 1.0 red was heated at about 50° C. Lipase (Novozym 435 from Candida antarctica), 5 g, was added to the fatty acid mixture. The fatty acids and enzyme were then subjected to a vacuum (1 to 10 mm Hg) and N₂ sparge. To the reaction was added propylene glycol (60 g) over the course of about 30 minutes. A sample of the reaction was taken at the times indicted in Table 2 below. The color values of the product after reaction were about 2.5 yellow and about 0.1 red.

TABLE 2
Production of PGME from Fatty Acids using Novozym 435

Sample Id

| | 119-7 | 119-8 | 119-9 | 119-10 |
|---|---|---|---|---|
| Time (min) | 30 | 60 | 90 | 120 |
| PGME | 65.66 | 66.88 | 66.78 | 66.99 |
| PGDE | 10.83 | 11.93 | 12.9 | 13.4 |
| FFA | 3.11 | 1.22 | 0.59 | 0.35 |
| PG | 37.32 | 36.52 | 36.53 | 35.68 |
| Total | 116.92 | 116.55 | 116.8 | 116.42 |

### Example 3

A mixture of fatty acid methyl esters (50 g) derived from soy having color values of about 0.6 yellow and about 0.1 red was heated at about 50° C. Lipase (Novozym 435), 5 g, was added to the fatty acid mixture. The fatty acids and enzyme were then subjected to a vacuum (1 to 10 mm Hg) and N₂ sparge. To the reaction was added propylene glycol (50 g) over the course of about 30 minutes. A sample of the reaction was taken at the times indicted in Table 3 below. The color values of the product after reaction were about 0.7 yellow and 0.1 red.

TABLE 3
Production of PGME from Fatty Acid
Methyl Esters using Novozym 435

Sample ID

| | 125-1 | 125-2 | 125-3 | 125-4 |
|---|---|---|---|---|
| Time | 30 | 60 | 90 | 120 |
| PGME | 60.93 | 66.94 | 68.42 | 69.21 |
| PGDE | 4.73 | 6.77 | 7.55 | 8.11 |
| FAME | 7.61 | 2.95 | 1.81 | 1.17 |
| PG | 29.72 | 30.44 | 28.09 | 30.83 |
| Total | 103.31 | 107.27 | 106.01 | 109.5 |

Having now fully described this invention, it will be understood to those of ordinary skill in the art that the same can be performed within a wide and equivalent range of conditions, formulations, and other parameters without affecting the scope of the invention or any embodiment thereof.

All documents, e.g., scientific publications, patents, patent applications and patent publications, recited herein are hereby incorporated by reference in their entirety to the same extent as if each individual document was specifically and individually indicated to be incorporated by reference in its entirety. Where the document cited only provides the first page of the document, the entire document is intended, including the remaining pages of the document.

What is claimed is:

1. A method for producing a propylene glycol monoester composition comprising:
(a) heating a fatty acid material at a temperature from about 10° C. to about 90° C.;
(b) contacting the heated fatty acid material with a lipase to form a mixture; and
(c) contacting said mixture with propylene glycol;
wherein a propylene glycol monoester composition is produced.

2. The method of claim 1, wherein said lipase is an immobilized lipase.

3. The method of claim 1, wherein said lipase is a 1,3-specific lipase.

4. The method of claim 1, wherein said lipase is derived from Mucor miehei or Candida antarctica.

5. The method of claim 1, wherein said lipase is Lipozyme RM IM or Novozym 435.

6. The method of claim 1, wherein said steps (a), (b) and (c) proceed essentially in the absence of an organic solvent.

7. The method of claim 1, wherein said fatty acid material is an admixture of fatty acid compounds derived from a vegetable oil.

8. The method of claim 7, wherein said vegetable oil is selected from the group consisting of soybean oil, linseed oil, sunflower oil, castor oil, corn oil, canola oil, rapeseed oil, palm kernel oil, cottonseed oil, peanut oil, coconut oil, palm oil, tung oil, safflower oil and derivatives, conjugated derivatives, genetically-modified derivatives and mixtures thereof.

9. The method of claim 1, wherein said fatty acid material comprises unsaturated fatty acids or derivatives thereof.

10. The method of claim 9, wherein said unsaturated fatty acids or derivatives thereof comprise unsaturated fatty acids selected from the group consisting of palmitoleic acid, oleic acid, linoleic acid, linolenic acid, eleostearic acid, ricinoleic acid, arachidonic acid, cetoleic acid, erucic acid and mixtures and derivatives thereof.

11. The method of claim 9, wherein said fatty acid material contains polyunsaturated fatty acids or derivatives thereof.

12. The method of claim 9, wherein said fatty acid material is a fatty acid C₁₋₄ alkyl ester.

13. The method of claim 12, wherein said C₁₋₄ alkyl ester is a methyl ester.

14. The method of claim 1, wherein said temperature is from about 30° C. to about 70° C.

15. The method of claim 14, wherein said temperature is from about 40° C. to about 60° C.

16. The method of claim 1, wherein said propylene glycol monoester composition comprises at least about 60% propylene glycol monoesters (PGMEs) by weight of the composition.

17. The method of claim 16, wherein said propylene glycol monoester composition comprises at least about 70% PGMEs by weight of the composition.

18. The method of claim 16, wherein said propylene glycol monoester composition further comprises less than about 15% propylene glycol diester by weight of the composition.

19. The method of claim 18, wherein said propylene glycol monoester composition further comprises less than about 5% propylene glycol diester by weight of the composition.

20. The method of claim 1, wherein said propylene glycol monoester composition has a Lovibond color less than or equal to about 3.5 yellow and about 1.0 red.

21. The method of claim 1, further comprising conducting step (b) while heating at a temperature from about 10° C. to about 90° C.

22. The method of claim 21, further comprising conducting step (c) while heating at a temperature from about 10° C. to about 90° C.

23. A method for producing a propylene glycol monoester composition comprising:
(a) contacting a fatty acid material with a lipase to form a first composition;
(b) contacting said first composition with propylene glycol to form a second composition; and
(c) heating said second composition at a temperature from about 10° C. to about 90° C.;
wherein a propylene glycol monoester composition is produced.
